(19) United States
(12) Patent Application Publication
(10) Pub. No.: US 2006/0233763 A1
(43) Pub. Date: Oct. 19, 2006

(54) Cartilaginous cell culture medium and the use thereof

(76) Inventor: Christine Aldrian, Freiburg (DE)

Correspondence Address: RATNERPRESTIA, P O BOX 980, VALLEY FORGE, PA, 19482-0980, US

(21) Appl. No.: 10/549,508

(22) PCT Filed: Mar. 17, 2004
(86) PCT No.: PCT/EP04/02753

§ 371 Date: Jun. 26, 2006

(30) Foreign Application Priority Data
Mar. 17, 2003 (DE) 10311620.6

(51) Int. Cl. C12N 5/08; A61K 35/32

(52) U.S. Cl. 424/93.7; 435/366

(57) ABSTRACT

A cartilage cell culture medium comprising a basic medium suitable for culturing primary human cells and, said culture medium additionally containing 0.5 to 50.0 ng/ml of EGF, 0.5 to 50.0 ng/ml of FGF, 0.1 to 10.0 ng/ml of PDGF, 0.5 to 50.0 ng/ml of IGF, 1.0 to 100.0 ng/ml of dexamethasone, and 0.1 to 10.0 mM of glutamine, optionally supplemented with 0 to 15% of autologous human serum. In a second aspect, the invention relates to the use of a cartilage cell culture medium as defined above for culturing cartilage cells (chondrocytes).

The present invention relates to a culture medium suitable for cartilage cells and preferably selective to cartilage cells.

### BACKGROUND OF THE INVENTION

Cartilage cells (chondrocytes) are cells responsible for forming cartilage tissue, especially in the joints. Injuries of the cartilage, for example as a result of accidents, or damages to the cartilage due to old age or wear generally affect mobility seriously and cause pain to those afflicted.

In the past, the only option open to physicians in cases of defective cartilage tissue was to immobilise the joint. In today's modern transplantation medicine, such defects can often be alleviated or eliminated by transplanting suitable tissue material.

In such a procedure, cells are usually incorporated into a three-dimensional carrier structure the shape of which usually corresponds to the shape of the future implant. The U.S. Pat. Nos. 5,053,050 and 5,736,372 describe compositions for repairing cartilage or bone, such cartilage or bone cells being incorporated into a carrier substance which may be resorbed biologically. In order to support the cell culture, substances promoting cell growth, adhesion factors, etc. may be added to the culture medium.

Joint cartilage replacements or cartilage transplants, respectively, consisting of a carrier material (for example, a non-woven material suitable for resorption), a biological matrix (for example, a gel) and autologous cells, especially chondrocytes, are usually produced by culturing the chondrocytes from the biopsate in a 3-D culture in an RPMI medium for approx. 7 weeks, embedding or incorporating e.g. by fulling the chondrocytes into the matrix, followed by 2 to 5 days of preculturing in the non-woven material. Each culture medium used for culturing the cartilage cells must be able to accomplish the following:

Firstly, it must be capable of promoting proliferation of the cartilage cells. Secondly, it should delay dedifferentiation of the chondrocytes into a fibroblastoid type as long as possible. Since dedifferentiation and strong proliferation are usually closely associated, it is necessary to find a suitable compromise between the properties promoting growth and those stabilising differentiation. Finally, a culture medium must be capable of maintaining the chondrogenic character of the cartilage cells.

At present, RPMI 1640 (containing glucose, amino acids, vitamins, salts and carbonate buffer) is generally used as a commercial product for medical purposes optionally supplemented with 10 to 15% of human serum. The latter must be obtained from a donor which places additional strain on this person. In general, the cells to be transplanted are cultured in this medium for approx. 7 weeks until the desired cell count has been reached. After seeding onto the transplant carrier, the cells must be cultured in the differentiation medium for a further 2 to 5 days until transplantation.

As a rule, an RPMI medium supplemented with FCS (usually 15%) is used as the culture medium for scientific purposes. Its use for medical purposes is ruled out.

Therefore, it is the object of the present invention to provide a cartilage cell culture medium which meets the above-mentioned criteria. It is another object of the present invention to provide a cartilage cell culture medium which permits shorter culturing times until the desired cell count has been reached and/or a shorter time span until seeding of the transplant carrier. Minimisation of the human serum content simplifies logistics and constitutes a substantial relief for the patient.

### SUMMARY OF THE INVENTION

The invention achieves the above-mentioned objects by a cartilage cell (chondrocyte) culture medium containing a basic medium suitable for culturing primary human cells which the following additional growth factors are added to: 0.5 to 50.0 ng/ml of EGF, 0.5 to 50.0 ng/ml of FGF, 0.1 to 10.0 ng/ml of PDGF, 0.5 to 50.0 ng/ml of IGF, 1.0 to 100.0 ng/ml of dexamethasone, and 0.1 to 10.0 mM of glutamine. If desired, this may be supplemented with 0 to 15% of human serum.

In a second aspect, the invention relates to the use of a cartilage cell culture medium as defined above for culturing cartilage cells.

### SHORT DESCRIPTION OF THE DRAWINGS

FIG. 1 shows the cell count after 8 days of culturing in different culture media obtained according to example 1 in the form of a bar chart.

FIG. 2 shows the time until the desired cell count is obtained by comparing three biopsates in a conventional medium (PRMI) and a medium of the invention.

### DETAILED DESCRIPTION OF THE INVENTION

As described above, the invention relates to a special cartilage cell culture medium as defined in the claims. Preferably, said cartilage cell culture medium exclusively contains substances permitted under the German Law on Pharmaceuticals and the guidelines of good medical practice (GMP) of the EU for use in humans. The cartilage cells (chondrocytes) recovered from said culture may therefore be used as a transplant without any concerns regarding the culture medium.

The cartilage cell culture medium contains a basic medium suitable for culturing primary human cells, which basic medium is preferably enriched and additionally contains: 0.5 to 50.0 ng/ml of EGF, 0.5 to 50.0 ng/ml of FGF, 0.1 to 10.0 ng/ml of PDGF, 0.5 to 50.0 ng/ml of IGF, 1.0 to 100.0 ng/ml of dexamethasone, and 0.1 to 10.0 mM of glutamine. Preferably, the cartilage cell culture medium additionally contains 5.0 to 20.0 ng/ml of EGF, 5.0 to 20.0 ng/ml of FGF, 1.0 to 6.0 ng/ml of PDGF, 5.0 to 20.0 ng/ml of IGF, 10.0 to 60.0 ng/ml of dexamethasone, and 1.0 to 6.0 mM of glutamine. Even more preferably, it contains 10.0 ng/ml of EGF, 10.0 ng/ml of FGF, 3.0 ng/ml of PDGF, 10.0 ng/ml of IGF, 40.0 ng/ml of dexamethasone, and 4.0 mM of glutamine.

According to the invention any known basic medium which is suitable for culturing primary human cells may be used. Preferably, this will be a comparatively rich medium (not a deficient medium). Said basic medium may use any suitable carbon and/or nitrogen source. Preferably, carbon and nitrogen sources corresponding to specification of the German Law on Pharmaceuticals and the GMP guidelines are used. Concentrations are variable in the present case and may be determined and optimised by the person skilled in the art with routine experiments. In addition, the medium preferably has a Ca²⁺ content of not more than 2.5 mM, preferably not more than 1.8 mM. Moreover, the Ca²⁺ content is preferably not below 0.4 mM, more preferably not below 0.6 mM.

Media which are suitable and preferred for the purposes of the present invention are selected from the group consisting of Eagle's MEM, DMEM, MEM, RPMI, and OptiPro. A particularly preferred medium is the basic medium OptiPro® which is sold by the Invitrogen company.

The first addition to the cartilage cell culture medium of the invention is the EFG (epidermal growth factor) in an amount of 0.5 to 50.0 ng/ml, preferably 5.0 to 20.0 ng/ml, most preferably 10.0 ng/ml. EGF is a general growth factor for mesenchymal cells. Preferably, it is recombinant, more preferably recombinant human EGF (rh EGF).

The second addition to the cartilage cell culture medium of the invention is the basic FGF (fibroblast growth factor) in an amount of 0.5 to 50.0 ng/ml, preferably 5.0 to 20.0 ng/ml, most preferably 10.0 ng/ml. In vivo, bFGF is a general growth factor for mesodermal and neuro-ectodermal cells. In vitro, bFGF lowers the demands concerning the serum quality. Preferably, it is a recombinant, more preferably a recombinant human bFGF.

The third addition to the cartilage cell culture medium of the invention is the PDGF (platelet derived growth factor) in an amount of 0.1 to 10.0 ng/ml, preferably 1.0 to 6.0 ng/ml, and most preferably 3.0 ng/ml. The generic term PDGF comprises a whole spectrum of related molecules which have different tasks. Among other things, PDGFs function as growth factors, in wound healing and as chemotactic factors. Preferably, a recombinant human PDGF (rh PDGF) is used. More preferably, these are BB homodimers.

The fourth addition of the cartilage cell culture medium of the invention is the IGF (insulin-like growth factor), preferably IGF-1, in an amount of 0.5 to 50.0 ng/ml, preferably 5.0 to 20.0 ng/ml, most preferably 10.0 ng/ml. In particular, IGF as a generic term designates IGF-1 and IGF-2. The IGFs are strong growth factors, among other things for cartilage cells. In part, they have a synergistic effect with other growth factors. Preferably, a recombinant, more preferably a recombinant human IGF (rh IGF) is used.

The fifth addition to the cartilage cell culture medium of the invention is dexamethasone in an amount of 1.0 to 100.0 ng/ml, preferably 10.0 to 60.0 ng/ml, most preferably 40.0 ng/ml. Dexamethasone (9α-fluoro-16α-methylprednisolone) is a semi-synthetic glucocorticoid which influences ACTH secretion. Dexamethasone is a differentiation factor for cartilage cells.

The sixth addition to the cartilage cell culture medium of the invention is glutamine in an amount of 0.1 to 10.0 mM, preferably 1.0 to 6.0 mM, most preferably about 4.0 mM. Glutamine is one of the amino acids occurring in nature which may generally serve as a nitrogen source.

In addition, the medium of the invention may, but need not be supplemented with human serum, preferably autologous, in an amount of 0 to 15%, preferably 0 to 10%, most preferably 3 to 5% (w/v). This shows that the culture medium of the invention permits an advantageous decrease of this added substance which was necessary in higher quantities in the prior art so far.

In a second aspect, the present invention relates to the use of the above-mentioned cartilage cell culture medium for culturing cartilage cells, especially the selective culturing thereof. The culturing conditions are known to the person skilled in the art and may be optimised by routine experimentation.

The culturing of cartilage cells using the medium of the invention starts with tissue biopsies of a donor, preferably the patient to be treated himself or herself. Preferably, the cartilage cells obtained are intended for reimplantation into a human, especially the donor. If the donor and the recipient are identical, there is no fear that the transplant will be rejected provided the culture medium does not contain antigenic substances. The latter does not comply with the German Law on Pharmaceuticals and is therefore not desirable.

A surprising and unexpected advantage of the medium of the invention with the combination of additions of the invention are the high proliferation rates of the cartilage cells in the example with only 3% of autologous serum. When carrying out comparative experiments with the commercial medium RPMI supplemented with human serum (10% and 15%), the applicant found that the invention cartilage culture medium provided better results with regard to growth rates. Specifically, the time until seeding of the transplant carrier and then until transplantation may be reduced noticeably by using the culture medium of the invention. By shorter culturing and optimised factors, the cells are less dedifferentiated. Thus, the chondrogenic potential and thus the quality of the product are enhanced. In a preferred embodiment, for example, this time span may be shortened by a factor of 3.5.

### EXAMPLES

### Example 1

In a first example, 0.1 million cells of one biopsate, i.e. of identical origin, were cultured in five different media and the cell count determined after 8 days in the culture. Culturing was conducted at 37° C. in cell culture bottles. For comparison, RPMI supplemented with 15% of human serum (HS) as the previous cartilage medium and the medium Optipro of Invitrogen and growth additives, supplemented with 3% HS, were used.

TABLE 1
Growth additives used (BTT factor mix)

| Addition | Amount | |
|---|---|---|
| EGF | 10 | ng/ml |
| FGF | 10 | ng/ml |
| PDF | 3 | ng/ml |
| IGF | 10 | ng/ml |
| Dexamethasone | 40 | ng/ml |
| Glutamine | 4 | mM |

TABLE 2
media tested

| Basic medium | Human serum (%) | BTT factor mix |
|---|---|---|
| RPMI | 3 | + |
| RPMI (comparison) | 15 | − |
| OptiPro ® | 3 | + |
| OptiPro ® | — | + |
| OptiPro ® (comparison) | 3 | − |

For direct comparison, parallel cell cultures of biopsates from the same test person were established with the media listed in table 2 and maintained according to common processes. As a result, the growth and the morphology of established cartilage cell cultures with the medium of the invention is approximately the same as that of the supplemented RPMI medium.

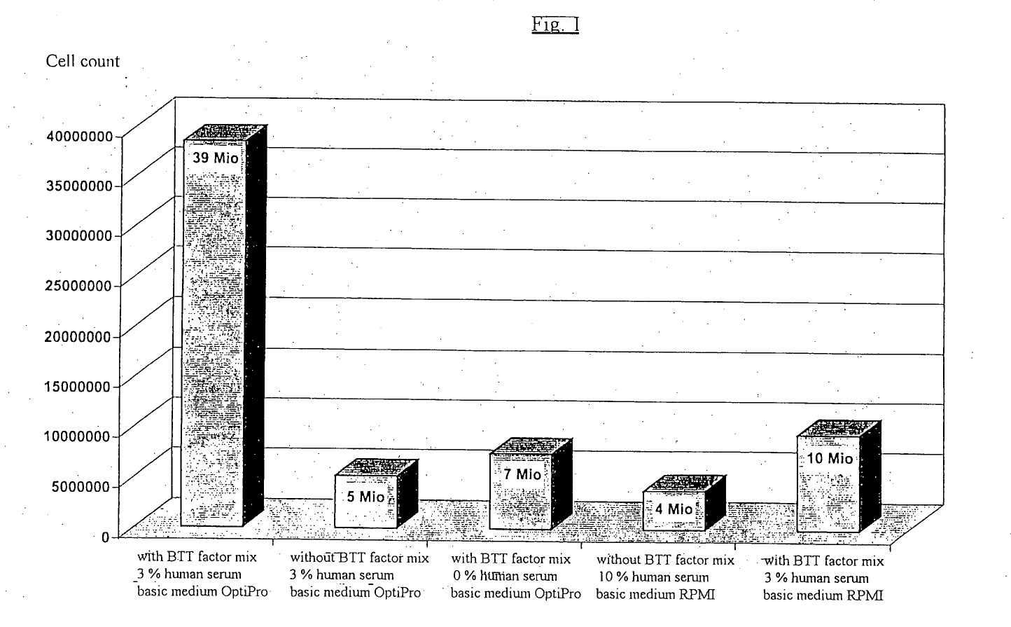

However, the medium of the invention offers advantages in establishing the culture to the effect that the cell yield with lower supplementation with HS is 2.5 to 10 times higher on average. The results are shown in FIG. 1.

At the same time, the time span of the culture and the number of the passages necessary until the cell count required for seeding the transplant is obtained may be reduced. On the whole, transplantation may be carried out earlier and with a better cell quality.

### Example 2

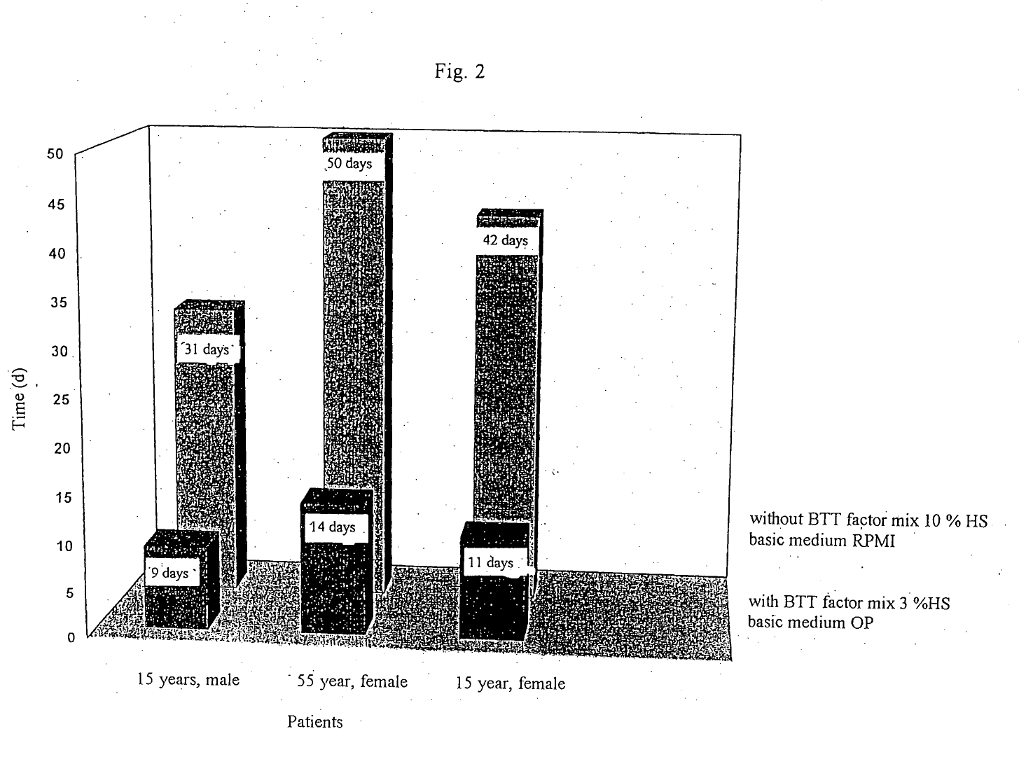

In a second experiment, the biopsates of three donors were cultured in the medium of the invention (No. 3 above; OptiPro® with 3% HS and additions) and RPMI (10% HS) and the time span until manifestation of the necessary cell count in the culture based on identical seeds determined. The results are shown in FIG. 2

FIG. 2 shows that the time span until a cell count of 60 mns. cells is obtained may be shortened considerably (approx. by a factor of 3, depending on the age of the donor) by culturing in the medium of the invention.

1. A cartilage cell culture medium comprising a basic medium suitable for culturing primary human cells and, additionally, 0.5 to 50 .ng/ml of EGF, 0.5 to 50.0 ng/ml of FGF, 0.1 to 10.0 ng/ml of PDGF, 0.5 to 50.0 ng/ml of IGF, 1.0 to 100.0 ng/ml of dexamethasone, and 0.1 to 10.0 mM of glutamine.

2. A cartilage cell culture medium according to claim 1 comprising 5.0 to 20.0 ng/ml of EGF, 5.0 to 20.0 ng/ml of FGF, 1.0 to 6.0 ng/ml of PDGF, 5.0 to 20.0 ng/ml of IGF, 10.0 to 60.0 ng/ml of dexamethasone, and 1.0 to 6.0 mM of glutamine.

3. A cartilage cell culture medium according to claim 1 comprising 10.0 ng/ml of EGF, 10.0 ng/ml of FGF, 3.0 ng/ml of PDGF, 10.0 ng/ml of IGF, 40.0 ng/ml of dexamethasone, and 4.0 mM of glutamine.

4. A cartilage cell culture medium as in claim 1, additionally comprising up to 15% (w/v) of human serum.

5-10. (canceled)

11. A cartilage cell culture medium as in claim 2, additionally comprising up to 15% (w/v) of human serum.

12. A cartilage cell culture medium as in claim 3, additionally comprising up to 15% (w/v) of human serum.

13. A cartilage cell culture medium as in claim 1, additionally comprising 3 to 5% (w/v) of human serum.

14. A cartilage cell culture medium as in claim 2, additionally comprising 3 to 5% (w/v) of human serum.

15. A cartilage cell culture medium as in claims 3, additionally comprising 3 to 5% (w/v) of human serum.

16. A cartilage cell culture medium according to claim 1, wherein the basic medium is selected from the group consisting of Eagle's MEM, DMEM, RPMI, MEM and OptiPro®.

17. A cartilage cell culture medium according to claim 2, wherein the basic medium is selected from the group consisting of Eagle's MEM, DMEM, RPMI, MEM and OptiPro®.

18. A cartilage cell culture medium according to claim 3, wherein the basic medium is selected from the group consisting of Eagle's MEM, DMEM, RPMI, MEM and OptiPro®.

19. A cartilage cell culture medium as in claim 16, additionally comprising up to 15% (w/v) of human serum.

20. A cartilage cell culture medium as in claim 17, additionally comprising up to 15% (w/v) of human serum.

21. A cartilage cell culture medium as in claim 18, additionally comprising up to 15% (w/v) of human serum.

22. A method of culturing cartilage cells obtained from tissue biopsies of a human donor comprising the step of growing said cartilage cells in a cartilage cell culture medium comprising a basic medium suitable for culturing primary human cells and, additionally, 0.5 to 50 .ng/ml of EGF, 0.5 to 50.0 ng/ml of FGF, 0.1 to 10.0 ng/ml of PDGF, 0.5 to 50.0 ng/ml of IGF, 1.0 to 100.0 ng/ml of dexamethasone, and 0.1 to 10.0 mM of glutamine.

23. The method of claim 22, wherein said cartilage cells are obtained from tissues biopsies performed on the patient in whom said cartilage cells are intended to be reimplanted.

24. The method of claim 22, wherein the cartilage cell count in said medium is determined for implantation into a human being.

25. The method of claim 22, wherein said cartilage cells are implanted into a human being.

26. The method of claim 23, wherein the cartilage cell count in said medium is determined for reimplantation into a human being.

27. The method of claim 23, wherein said cartilage cells are reimplanted into a human being.

28. A cartilage cell suspension in a medium as claimed in claim 1.
